(19) United States
(12) Patent Application Publication
(10) Pub. No.: US 2007/0196419 A1
(43) Pub. Date: Aug. 23, 2007

(54) Composite Materials Based On Polysilicic Acid And Method For The Production Thereof

(76) Inventors: Marianne Teller, Mistorf (DE); Hans-Georg Neumann, Rostock (DE)

Correspondence Address: JORDAN AND HAMBURG LLP, 122 EAST 42ND STREET, SUITE 4000, NEW YORK, NY, 10168, US

(21) Appl. No.: 10/592,853

(22) PCT Filed: Mar. 7, 2005
(86) PCT No.: PCT/EP05/51012

§ 371 Date: Jan. 11, 2007

(30) Foreign Application Priority Data
Mar. 13, 2004 (DE) 102004012411.6

(51) Int. Cl. A61K 9/32

(52) U.S. Cl. 424/423; 977/906; 424/482

(57) ABSTRACT

The invention relates to composite materials based on polysilicic acid, said materials containing novel compositions which have improved material properties and can be in the form of dispersions, pastes, powders, granulated materials, layers or compact moulded bodies. The aim of the invention is to produce composite materials based on polysilicic acid with improved mechanical properties. To this end, the composite materials contain polysilicic acid, between 0.01 and 20 mass % of an organic polymer, more than 15 mass % of at least one calcium phosphate phase, and optionally a use-specific additive. The material produced according to the invention can be implanted or injected. The composition of the composite material with the resulting properties enables the composite material to be used for bone substitution and/or bone regeneration in both human medicine and animal medicine. The inventive material can also be used to heal wounds.

### BACKGROUND OF THE INVENTION

The invention relates to composite materials based on polysilicic acid that contain an organic polymer, at least one calcium phosphate phase, and optionally application-specific additives as additional components, and that can be present in the form of dispersions, pastes, powders, granulates, layers, or other compact molded bodies.

There are already applications or potential application areas for such composite materials in the fields of human medicine and veterinary medicine, especially in the use of bone replacement or bone regeneration material or as a coating on orthopedic, trauma, tological or dental implants.

It is the nature of composite materials that the properties related to the individual components together have an effect. This combination effect e.g. under physiological conditions leads to the fact that both inorganic and organic components of the composite material can be generated or used as inorganic and organic metabolism products.

The composite materials known in this context comprise either polysilicic acid and at least one calcium phosphate phase or at least one calcium phosphate phase and one organic polymer. Thus DE10003824A1 claims a bone replacement material that includes porous silicon dioxide and calcium phosphate composites that are shaped into filaments via nozzles.

Composites that contain silicon dioxide and calcium phosphate chemical components have also been described as bioglass, bioactive glass, bioceramics, and bioactive ceramics. WO9317976, WO9404657, WO9514127, and WO9636368 describe materials that contain the aforesaid components and that are provided for use as bone replacement materials or as templates for the synthesis of bone tissue.

Adding ceramic and polymer fibers has also been suggested for structuring such composites (U.S. Pat. No. 5,468,544). Moreover, it is also possible to integrate biologically active molecules into these composite structures and release them in a controlled manner (U.S. Pat. No. 5,874,109).

Moreover, U.S. Pat. No. 6,416,774 describes a material that comprises nanoporous calcium phosphate particles that contain portions of silicon dioxide and biologically active components.

Various compositions and options for producing them have been described for producing composite materials made of calcium phosphate phases and physiologically relevant organic polymers. DE68928975T2 describes composites that contain a calcium phosphate phase, a tannin derivative, and a collagen compound.

DE4132331C2 describes a calcium phosphate cement powder that contains a water-soluble polymer in addition to calcium phosphate phases. DE69809158T2 embodies the combination of calcium phosphate powders with a polysaccharide.

DE19956503 A1 discloses a bone replacement material that contains a hardened matrix, e.g. in the form of polyglycolic-co-lactic acid and other organic components and viable cells in addition to a calcium phosphate phase. The bone replacement material produced is prepared in a multiple syringe comprising a plurality of syringes that are combined or in a complete syringe with a plurality of chambers.

WO9911296 describes an osteosynthetic composite material containing 3 components. The components comprise a bioceramic or a bioglass, a biologically degradable polymer, and a biologically degradable polymer matrix.

In Biomaterials 2002, 23 (15), 3227-34, Zhao et. al. describe the production of a three-dimensional organic network in which hydroxyapatite granules are distributed in a chitosan/gelatin mixture.

In CN1338315, solutions containing phosphate and calcium are dripped together with sodium hydroxide into an acid solution of collagen; at the end of the procedure the product is separated by centrifuging and ground. The sequence is changed in CN 1337271. In this case, a solution of calcium ions is added to an acid collagen solution, then added to a phosphate solution drop by drop, and finally the pH is adjusted with sodium hydroxide.

The Collagraft Strip, a product from NeuColl that is on the market, is based on a composition of hydroxyapatite (65%), tricalcium phosphate (35%), and ultrapure collagen type 1. The material described is compared to autogenous bone and evaluated positively (see http://www.neucoll.com).

Bonfield et. al. describe a phase diagram to calcium phosphate collagen systems using temperature and pH (Bioceramics, Vol. 16, eds. M. A. Barbosa et. al., Trans Tech Publications Ltd., Uetikon Zurich, 2003, pp. 593-596).

As a rule the composite materials described in the foregoing are produced by simultaneous or temporally staggered mixing procedures. In contrast thereto, WO02059395A2 claims electrochemical deposition of calcium phosphate and chitosan from electrolytes that contain corresponding components and precursors.

In DE10029520A1, Scharnweber et. al. produce a bone-like coating that is generated biomimetically. The mineralized collagen matrix is constructed by layer. The organic phase is applied by dipping into a collagen solution and then the calcium phosphate phases are deposited using an electrochemically supported process.

Finally, it should be mentioned that bioactive glasses are converted to polymer suspensions for the purpose of injectability (WO0030561).

The items listed in the following are disadvantages of all of the aforesaid methods.

1. Usable composite materials can only be generated from two base materials, either silicon dioxide in combination with calcium phosphate (or other components in bioglasses) or calcium phosphate with physiologically compatible polymers.

2. Composite materials based on bioglasses and related materials have a phosphate content (as P₂O₅) of only less than 15%.

3. The mechanical properties of the composites are determined by the main components. Thus in the combination of silicon dioxide and calcium phosphate the composite is almost totally non-elastic. On the contrary, the materials produced at low temperatures are brittle and the materials produced at high temperatures are characterized by great hardness.

4. As a rule, the temperatures used during the production or post-treatment of composites (500-1200° C.), which temperatures are absolutely necessary for the physical/chemical structuring of the materials, do not permit direct integration of organic polymers, especially bioorganic polymers.

Thus the object underlying the present invention is to make accessible composite materials based on polysilicic acid having improved mechanical properties and that contain a plurality of physiologically relevant inorganic and organic components.

The structuring of the composite materials should be largely variable and should contain liquid, paste-like, and solid shapes. Bone replacement matter, bone regeneration matter, and

bone cements should be realized with composite materials.

The composite materials should also be able to be used for coating implant surfaces regardless of their material compositions and surface structures.

### SUMMARY OF THE INVENTION

This object, to produce composite materials based on polysilicic acid as a surface layer on human or veterinary medical or as bone replacement materials or bone regeneration materials with improved properties, is inventively attained in that the composite materials contain polysilicic acid, an organic polymer, at least one calcium phosphate phase, and optionally an application-specific additive in specific ratios. It has been demonstrated that a content of calcium phosphate materials of greater than 15 mass % effects a significant increase in the physical/chemical stability of the composite. Depending on its chemical structure and the associated properties, the organic polymer should be in the range of 0.01 to 20 mass % with respect to the other composite components and to the applicable requirements.

The polysilicic acid can be generated from various sources that can be used alone or in combination. For instance, a condensation of inorganic silicates, which are generally embodied under acid or neutral pH conditions, is possible, whereby the polysilicic acid matrix can contain proportionally other metal oxides, such as titanium dioxide and aluminum oxide or their precursors. Tetraalkoxysilanes or organoalkoxysilanes can also be used as starting materials for forming polysilicic acid and corresponding derivatives. Another source can be found in solid spherical or amorphous nano- or micro-silicate particles, whereby the chemical functionality required for composite formation is produced using the properties of the particle surface. For reasons related to production and application, the particle diameter used is preferably between 10 nm and 10 μm.

The addition of organic polymer effects a positive change in the elasticity of the composite material. The composite produced is more compression-elastic and thus less brittle. Natural, synthetic, or semisynthetic polymers can function as organic polymers. They are used in the form of homopolymers or copolymers or even as polymer blends that have naturally or synthetically added reactive and functional groups, sequences, or substructures.

From an application engineering perspective, the use of biopolymers as composite component is preferred. This relates in particular to proteins and polysaccharides, their fragments and derivatives, such as e.g. celluloses, laminars, starch, or its components amyloses and amylopectin, glycogen, dextrines, dextran, pullulan, inulin, chitosan, xanthan, alginic acid and its salts and esters, gum arabic, chondroitin, heparin, and keratan as well as sulfates derived therefrom, hyaluronic acid and teichoic acids, and esters, collagen and gelatins derived therefrom in native or modified form.

Likewise, synthetic polymers that have adequate compatibility with all of the media involved can be used. This applies to their use alone as well as in combination with biopolymers. Such synthetic polymers derive in particular from the polyamine and polyimine compound classes, polyols and their ethers and esters, polycarboxylic acids including derivatives thereof such as esters and amides. In principle, however, other polyvinyl compounds, polyethers, polyesters, polyketones or polysulfones can be considered as a composite component.

In particular solubility in water or in the selected reaction medium, swellability, or dispersability of the organic polymers determine the percentage portion in the composite material (0.01% and 20% (w/w)).

Apart from polysilicic acid, as a rule the main component of the composite material is at least one calcium phosphate phase. This calcium phosphate phase can be added to the reaction medium preproduced in crystalline or amorphous form, or it is prepared in situ in that calcium- and phosphate-containing components are combined under neutral or alkaline conditions. The calcium phosphate phase retains its morphology during the further production process so that a corresponding porosity is thus imparted to the final product. Hydroxyapatite, a- or b-tricalcium phosphonate, dicalcium phosphate, dicalcium phosphate dihydrate, octacalcium pentaphosphate, or corresponding mixed phases or mixtures, among other things, can be used for the calcium phosphate phase. From a therapeutic perspective, it has proved useful for the calcium phosphate phase in certain cases also to contain a portion of alkaline bisphosphonate calcium salts. The calcium phosphate phase can additionally be effective as a calcium precursor for a complex formation with the polymer component of the composite. This complex formation is also intensified by the addition of alkaline bisphosphonate calcium salts. In addition, portions of other metal cations can be contained in the calcium phosphate phase, such as sodium, potassium, silver, magnesium, zinc, or lithium, as well as fluoride, chloride, sulfate, carbonate, or silicate anions.

An application-specific additive can optionally be added to the composite material in addition to the basic components of polysilicic acid, polymer, and calcium phosphate component. This additive can be used for a chemically or morphologically modified polysilicic acid compound, organic polymer, or calcium phosphate phase. Likewise, it is also possible to add an additive in the form of solid nano- or micro-particles or capsules. In addition to directly adding bioactive substances, such as for instance antibiotics, tumorstatic agents, hormones, or growth factors, or a combination of these substance classes, the bioactive substances can also be used in a capsule. Controlled release of the active substances can be effected in a known manner using the type and method of encapsulation.

The production engineering implementation of the production of the composite material begins in that an organic polymer, at least one calcium phosphate phase, and optionally an application-specific additive are added to a gel produced from silicic acid sol. The production of a gel starting with a silicic acid sol is described in detail in the literature (H. Schmidt: “Chemistry of Material Preparation by Sol-Gel Process” in J. Non-Cryst. Solids 100, 51 (1988); J; D. F. Ramsay: “Sol-Gel Processing” in Controlled Particle, proplett [sic] and Bubble Formation, Ed.: D. J. Wedlock Butterwoth-Heinemann Ltd., Oxford, 1994, pp. 1-36). The individual components of the composite material are combined successively or in combination depending on their chemical and physical properties. If all of the components are combined, various stirring techniques are used for homogenization depending on the viscosity of the mass (stirrers, dispergators). The viscosity also determines subsequent molding. While still moist, the composite material is, e.g., poured, pressed, injected, or even sprayed. Improved adhesion to surfaces can be achieved in that the composite material is deposited while moist and while reducing pressure. In addition, the composite material can be applied to surfaces by dipping or spraying. Application is not limited only to static surfaces, but rather can extend to rotating substrates.

Metal, natural, and synthetic or ceramic surfaces are suitable for coating with the composite matter, regardless of their roughness, pre-treatment, or prior coating.

Electrochemical (cathodic) deposition offers an entirely different option for coating with the composite material. In principle there are two options. Either the finished component mixture is used or the components are deposited electrochemically one after the other.

Combining polysilicic acid derivatives with appropriate calcium phosphates and polymers with the alternative of adding application-specific additives makes it possible to use the composite material in conjunction with medical products for directly as a medical product. The composite matter can be used directly as a base material, filler, depot material, or as a coating. The composite matter can be used in the form of dispersions, pastes, powders, granulates, layers, or even as compact molded bodies.

Due to the possibility that the composite matter itself can contain an application-specific additive, it can be used directly as a pharmaceutical or in combination with pharmaceuticals.

The material produced in accordance with the invention is implantable or injectable. The composition of the composite material with the resultant properties makes it possible to use the composite matter for bone substitution and/or for bone regeneration. Moreover, this material can be used for healing wounds.

The invention shall be described in greater detail using the following exemplary embodiments without being limited thereto.

### DETAILED DESCRIPTION OF THE INVENTION

### Example 1—Production of a Composite Material Based on Polysilicic Acid, Polymer, and a Calcium Phosphate Phase

3 ml 0.1 M hydrochloric acid and 3 ml ethanol are added to 9 ml tetraethoxylsilane. The hydrolyzate is stirred into 3 ml 1.5% chitosan solution (2% lactic acid) so that a clear solution is obtained. Then 9 g hydroxyapatite are added by means of a dispergator. After a reaction period of 2 h at 50° C. the matter can be pressed. Drying is performed at 100° C. The porosity of the material is 70% (determined using Archimedes' principle). Internal surface area determined by gas absorption is 120 m²/g. 44% of the pores are in the range of 20-80 nm. 18% of the pores have a diameter >80 nm.

### Example 2—Production of the Composite Material Based on Polysilicic Acid, Polymer, and to Calcium Phosphate Phases

3 ml 0.1 M hydrochloric acid and 3 ml ethanol are added to 8 ml aminopropyl trimethoxysilane. After hydrolysis has concluded, the polysilicic acid solution is added drop by drop, while stirring, to 5 ml 0.5% collagen solution (in 10% lactic acid). Then 6 g β-tricalcium phosphate and 12 g hydroxyapatite are stirred in one after another. The composite material is immediately molded as desired. After another reaction period of 2 h at 50° C., the composite material is dried at 100° C. The material has an internal surface area of 138 m²/g. 46% of the pores are in the range of 20-80 nm.

### Example 3-Production of a Composite Material Based on Polysilicic Acid, Polylactic Acid, and Hydroxyapatite—10% Polymer Part (w/w)

4 ml 0.1 M hydrochloric acid and 5.5 ml ethanol and 4 ml water are added to 13.8 ml tetraethoxysilane. 2 g polylactic acid (poly-D,L-lactide), inherent viscosity 0.16-0.24 dl/g, mean molecular weight 2000 g/mol) and 14 g hydroxyapatite are mixed homogenously and dispersed within 3 min into the polysilicic acid sol that has been cooled to the 10° C. The homogeneous mass is kept at 50° C. for about 2 h. Then it can be molded by pressing. The composite matter is left to cure at room temperature for 24 h and is then dried at 100° C.

### Example 4—Production of the Composite Material Based on Polysilicic Acid, Polylactic Acid, and Hydroxyapatite—20% Polymer Part (w/w)

4 ml 0.1 M hydrochloric acid and 5.5 ml ethanol and 4 ml water are added to 13.8 ml tetraethyoxysilane. 4 g polylactic acid (poly-D,L-lactide), inherent viscosity 0.16-0.24 dl/g, mean molecular weight 2000 g/mol) and 12.3 g hydroxyapatite are mixed homogenously and dispersed within 3 min into the polysilicic acid that has been cooled to 10° C. The homogeneous mass is kept at 50° C. for about 2 h. Then it can be molded by pressing. The composite matter is left to cure at room temperature for 24 h and is then dried at 100° C.

### Example 5—Production of a Composite Material Based on Polysilicic Acid, Active Substance-Containing Polylactic Acid Microparticles, and Hydroxyapatite

4 ml 0.1 M hydrochloric acid and 5.5 ml ethanol and 4 ml water are added to 14 ml tetraethoxysilane. 2 g poly(lactic acid-co-glycolic acid) microparticles (produced from poly(D,L-lactide-co-glycolide) (inherent viscosity 0.16-0.24 dl/g, mean molecular weight 17,000 g/mol), d=48 μm), that contain 20% vancomycin hydrochloride, and 14 g hydroxyapatite are mixed homogenously and dispersed 3 min into the polysilicic acid sol that has been cooled to 10° C. The homogeneous mass is kept at 50° C. for about 2 h. Then it can be molded by pressing. The composite matter is left to cure at room temperature for 24 h and is then dried at 100° C.

### Example 6—Production of a Composite Material Using Polysilicic Acid Nanoparticles

14 ml 0.1 M hydrochloric acid are added to 36 g colloid-disperse polysilicic acid nanoparticles (34 mass % SiO₂, surface area 110-150 m²g) and activated for 10 min in the ultrasonic bath. The solution is stirred into 15 ml chitosan solution (1.5% in 2% lactic acid). Then 11.9 g hydroxyapatite are dispersed into the white homogeneous solution. After a 1-h incubation period at 50° C., the composite material is molded by pressing or coating. The material is dried at 100° C.

### Example 7—Production of a Composite Material Using Polysilicic Acid Microparticles

3 g polysilicic acid microparticles (nonporous, plain, 7 μmol/g Si—OH, d=1 μm) are suspended in 8 ml 0.1 M hydrochloric acid. After an activation period of 10 min in the ultrasound bath, 15 g hydroxyapatite are stirred in until a homogeneous consistency is attained. The pressed mass is dried for 24 h in air and then dried at 100° C.

### Example 8—Production of a Composite Material Using Epoxy-Functionalized Polysilicic Acid Particles

1 g polysilicic acid nanoparticles (nonporous, epoxy-functionalized, spherical, 8 μmol/g, d=300 nm) are resuspended in 15 ml 2% sodium alginate solution by means of ultrasound. The solution is stirred for 15 min. Then 9.5 g hydroxyapatite are added for the calcium phosphate phase. The mass is kept at 50° C. for 1 h and is then molded as desired. The composite material is dried at 100° C.

### Example 9—Production of an Injectable Composite Material Starting with Polysilicic Acid, Polymer, and a Calcium Phosphate Phase

For producing an injectable composite material, a polysilicic acid sol is produced from 9 ml tetraethyoxysilane, 3 ml 0.1 M hydrochloric acid, and 3 ml ethanol water. 3 ml 1.5% chitosan solution (in 2% lactic acid) are added drop by drop. 10 ml of the polysilicic acid sol/chitosan solution are caused to react with 15.6 g dicalcium phosphate dihydrate via a mixing tip. The composite material produced in this manner does not dissolve when injected in SBF buffer and possesses compression-elastic properties.

### Example 10—Production of Composite Materials Using Successive Electrochemical Deposition of the Components

Chitosan is electrochemically deposited from a 1.5% chitosan solution, pH 5.0, on an electrochemically deposited calcium phosphate phase (composite from calcium phosphate phases that dissolve with difficulty and that dissolve easily). The excess gel-like chitosan film is rinsed off. Then a silicate surface is produced thereon in that a 0.1 M sodium silicate solution is used as polysilicic acid precursor. With the addition of 0.1 M calcium chloride solution and 1 M hydrochloric acid, the calcium-containing polysilicic acid layer is produced on the calcium phosphate/chitosan coating at a voltage between 5 and 8 V.

### Example 11—Production of Composite Material with Gentamicin Sulfate as Additive

8 ml 0.1 M hydrochloric acid and 10 ml ethanol are added to 27 ml tetraethoxysilane. After hydrolysis of the alkoxysilane has concluded, the polysilicic acid solution is added drop by drop to 7.5 ml collagen solution (in 10% lactic acid). 27 g hydroxyapatite and 18 g β-tricalcium phosphate are added to the solution while stirring. 2.25 g gentamicin sulfate are dissolved in 4 ml water and stirred into the composite material. After 30 min the product is poured into molds and dried at 130° C.

### Example 12—Production of Composite Material with Vancomycin Hydrochloride as Additive

5 ml 0.1 M hydrochloric acid are added to 18 ml tetraethoxysilane and then 7 ml ethanol are added. After hydrolysis has concluded, this solution is added to 5 ml 0.5% collagen solution (in 10% lactic acid). Then the hydroxyapatite (18 g) and β-tricalcium phosphate (12 g) calcium phosphate phases are stirred in. 1.5 g vancomycin hydrochloride are added as a solid and the composite material is stirred for 30 minutes using a magnetic stirrer. The composite material is poured into molds and then dried at 100° C.

### Example 13—Production of Composite Material with the Addition of Alkylenebisphosphonate

5 ml 0.1 M hydrochloric acid are added to 18 ml tetraethyoxysilane and then 7 ml ethanol are added. After hydrolysis has concluded, the solution is added to 5 ml 0.5% collagen solution (in 10% lactic acid). Then the hydroxyapatite (20 g) and β-tricalcium phosphate (10 g) calcium phosphate phases are stirred in. 1.0 g sodium clodronate (dichloromethylene-diphosphonic acid disodium salt) are added to 5 ml 1 M calcium chloride solution. Then this suspension is added to the composite material. The solution is stirred for 30 minutes. The composite material is poured into molds and then dried at 100° C.

1.-42. (canceled)

43. A composite material in a form selected from the group consisting of surface coatings on human and veterinary implants, bone replacement materials and bone regeneration materials, comprising a polysilicic acid matrix, at least one organic polymer in a weight proportion of 0.01 to 20% of the composite material and at least one calcium phosphate phase in a weight proportion greater than 15% of the composite material.

44. A porous composite material in accordance with claim 43, wherein the porous composite material is formed by providing at least one liquid carrier medium and drying a resultant composition comprising the polycilicic acid, the at least one organic polymer, the calcium phosphate phase and the carrier medium at a temperature below 150° C.

45. A porous composite material in accordance with claim 43, further comprising at least one additive.

46. A porous composite material in accordance with claim 43, wherein said polysilicic acid matrix is obtained by condensation from inorganic silicates.

47. A porous composite material in accordance with claim 43, wherein said polysilicic acid matrix is obtained by condensation from tetraalkoxysilanes.

48. A porous composite material in accordance with claim 43, wherein said polysilicic acid matrix comprises solid spherical or amorphous nano- or micro-silicate particles.

49. A porous composite material in accordance with claim 48, wherein said polysilicic acid matrix comprises solid spherical or amorphous nano- or micro-silicate particles, of diameters in the range of 10 nm to 10 μm.

50. A porous composite material in accordance with claim 43, wherein said polysilicic acid matrix is modified with organic groups by condensation of organoalkoxysilanes.

51. A porous composite material in accordance with claim 43, wherein said polysilicic acid matrix contains titanium dioxide and/or aluminum dioxide or their precursors.

52. A porous composite material in accordance with claim 43, wherein said at least one organic polymer is natural, synthetic, or semisynthetic.

53. A porous composite material in accordance with claim 43, wherein said at least one organic polymer comprises a homopolymer or copolymer or a polymer blend.

54. A porous composite material in accordance with claim 43, wherein said at least one organic polymer is derived using reactive or functional groups, sequences, or substructures.

55. A porous composite material in accordance with claim 43, wherein said at least one organic polymer is water-soluble or dispersible in water.

56. A porous composite material in accordance with claim 43, wherein said at least one organic polymer comprises at least one biopolymer.

57. A porous composite material in accordance with claim 56, wherein said at least one biopolymer is selected from the group consisting of polyamino acids, polypeptides, proteins, and fragments and derivatives thereof.

58. A porous composite material in accordance with claim 56, wherein said at least one biopolymer is selected from the group consisting of polysaccharides and fragments and derivatives thereof.

59. A porous composite material in accordance with claim 43, wherein said at least one organic polymer comprises a synthetic polymer selected from the group consisting of polyamines, polyimines, polyols and esters thereof, polycarboxylic acids and derivatives thereof, and polyvinyls.

60. A porous composite material in accordance with claim 43, wherein said at least one calcium phosphate phase comprises a preproduced calcium phosphate phase and/or a calcium phosphate phase prepared in situ.

61. A porous composite material in accordance with claim 43, wherein said at least one calcium phosphate phase comprises at least one phase comprised of a calcium phosphate selected from the group consisting of hydroxyapatite, alpha-tricalcium phosphonate, β-tricalcium phosphonate, dicalcium phosphate, dicalcium phosphate dihydrate, octacalcium pentaphosphate, and mixtures thereof.

62. A porous composite material in accordance with claim 43 wherein said at least one calcium phosphase comprises at least one alkylenebisphosphonate calcium salt.

63. A porous composite material in accordance with claim 43, wherein said calcium phosphate phase further comprises at least one metal cation selected from the group consisting of sodium, potassium, silver, magnesium, zinc and lithium cations.

64. A porous composition material in accordance with claim 43, wherein said calcium phosphate phase further comprises at least one anion as selected from the group consisting of fluoride, chloride, sulfate, carbonate and silicate anions.

65. A porous composite material in accordance with claim 45, wherein said at least one additive is selected from the group consisting of chemically or morphologically modified polysilicic acid compounds, additional organic polymers and additional calcium phosphate phases.

66. A porous composite material in accordance with claim 45, wherein said at least one additive comprises solid nano- or micro-particles or -capsules.

67. A porous composite material in accordance with claim 45, wherein said at least one additive comprises bioactive substances selected from the group consisting of antibiotics, tumorstatic agents, hormones, growth factors, and combinations thereof.

68. A porous composite material in accordance with claim 65, wherein said bioactive substances are contained in capsules for time release.

69. Method for producing a composite material comprising polysilicic acid, the method comprising dispersing an organic polymer and a calcium phosphate phase in a gel produced from silicic acid sol.

70. Method for producing a composite material in accordance with claim 69, further comprising adding an additive to the gel.

71. Method for producing a composite material in accordance with claim 69, wherein said organic polymer and said calcium phosphate phase are homogenously dispersed in said gel by stirring.

72. Method for producing a composite material in accordance with claim 69, further comprising molding the dispersion by pouring, pressing, injecting, or spraying the dispersion.

73. Method for producing a composite material in accordance with claim 69, further comprising depositing the dispersion on a surface.

74. Method for producing a composite material in accordance with claim 73, wherein said depositing comprises dipping or spraying the dispersion on the surface.

75. Method for producing a composite material in accordance with claim 73, wherein said surface is a surface of a rotating substrate.

76. Method for producing a composite material in accordance with claim 73, wherein said surface is of metal, a natural or synthetic polymer or a ceramic.

77. Method for producing a composite material in accordance with claim 73, wherein said depositing is electrochemical.

78. Method for producing a composite material in accordance with claim 73, further comprising repeating said depositing at least once.

79. A composite material produced by the method of claim 69.

80. A composite material in accordance with claim 79 in a form selected from the group consisting of base materials, fillers, depot materials and coatings.

81. A composite material in accordance with claim 79 in the form of dispersions, pastes, powders, granulates, layers and compacted molded bodies.

82. A composite material in accordance with claim 79, further comprising at least one bioactive compound and/or pharmaceutical.

83. A composite material in accordance with claim 79 in implantable or injectable form.

84. A method of using a composite material in accordance with claim 79, comprising applying the composite material to a medical or veterinary patient as a bone substitute and/or bone regenerator.

85. A method of using a composite material in accordance with claim 79, comprising applying the composite material to a wounded medical or veterinary patient for healing the wound.
